(19) United States
(12) Patent Application Publication
(10) Pub. No.: US 2011/0263909 A1
(43) Pub. Date: Oct. 27, 2011

(54) Method For Reprocessing Aircraft De-Icing Agents Comprising Glycol

(75) Inventors: Achim Stankowiak, Altoetting (DE); Ingo Jeschke, Tittmoning-Asten (DE); Thomas Bergstroem, Oberding (DE)

(73) Assignee: CLARIANT FINANCE (BVI) LIMITED, Tortola (VG)

(21) Appl. No.: 13/141,893

(22) PCT Filed: Dec. 2, 2009
(86) PCT No.: PCT/EP09/08584

§ 371 Date: Jun. 23, 2011

(30) Foreign Application Priority Data
Dec. 24, 2008 (DE) 10 2008 063 095.0
Dec. 2, 2009 (EP) PCT/EP2009/008584

(51) Int. Cl. C07C 29/80

(52) U.S. Cl. 568/868

(57) ABSTRACT

The invention relates to a method for reprocessing aircraft de-icing agents comprising glycol, wherein (1) the used aircraft de-icing agents are collected in a suitable device, (2) the used aircraft de-icing agent is subsequently brought to a propylene glycol content of between 55 and 75% by weight without prior separating of solid or suspended impurities by expelling water at increased temperature, (3) the concentrated used aircraft de-icing agent thus obtained is transported to a central reprocessing system, where the same is subjected to a fine distillation, and wherein (4) propylene glycol is produced as a distillate of the fine distillation.

The invention relates to a method for reprocessing used aircraft de-icing agents based on glycols.

Aircraft de-icing agents based on glycols are described for example in U.S. Pat. No. 4,358,389 and U.S. Pat. No. 4,744,913. They generally comprise
(a) about 40 to 80% by weight of at least one glycol having 2 or 3 carbon atoms or of a diglycol having 4 to 6 carbon atoms, for example ethylene glycol, diethylene glycol, propylene glycol and the like,
(b) 0.05 to 1.5% by weight of at least one polymeric component as thickener, for example from the group of polyacrylates, polymethacrylates, xanthan gum and cellulose derivatives,
(c) 0.05 to 1% by weight of at least one surfactant, for example olefinsulfonates, alkylarylsulfonates, polyoxalkylates and the like,
(d) at least one corrosion inhibitor in an effective amount, for example from he group of triazoles, imidazoles and/or phosphoric acid esters, and
(e) at least one basic compound for adjusting the pH to from about 7.5 to 11 and
(f) water as remainder to 100% by weight.

These aircraft de-icing agents are applied to the parts of the aircraft to be treated as they are (i.e. as concentrate) or following dilution with water for their preservation and/or for the freeing of ice and/or snow. The de-icing agent, which is now more or less diluted with melt water and contaminated with sand, rubber dust, oil, combustion residues and the like, flows from the treated parts of the aircraft into a collecting vessel and is referred to as wastewater from the aircraft de-icing or as used aircraft de-icing agent.

The used aircraft de-icing agents are sometimes disposed of with the help of a biological wastewater treatment plant. However—despite the good biodegradability of glycols—this leads to an undesired burden on the wastewater treatment plant, especially at low outside temperatures and reduced bacteria activity associated therewith, which is generally the case when using de-icing agents. A further disadvantage of this type of disposal of aircraft de-icing agents is the loss of the large amount of valuable glycol.

EP-A-0 637 620 discloses a method for reprocessing used aircraft dicing agents based on glycol, in which
(1) the used aircraft de-icing agent is firstly filtered to separate off the suspended impurities,
(2) the filtrate obtained in step (1) is subjected to an ultrafiltration to separate off the polymeric thickeners,
(3) the permeate obtained in step (2) is subjected to ultrafiltration with an anion exchanger and a cation exchanger to separate off any salts and ionic compounds present, and
(4) the solution obtained in step (3) is distilled to the desired value to remove excess water and thus adjust the glycol content,
(5) the glycol/water mixture obtained is supplied with suitable additives for use as aircraft de-icing agent.

EP-A-1 889 658 discloses a method for reprocessing glycol-containing aircraft de-icing agents in which they are subjected directly to a separation by means of a membrane, thus excluding a distillation in a subsequent method step. This demanding and complex method in terms of plant technology for reusing used aircraft de-icing agents only makes sense at airports which have a large demand for aircraft de-icing agent.

The object of the invention is to propose a simple and cost-effective method which makes it possible to largely recover and reuse in particular the glycols from used aircraft de-icing agents, and where the reusability of the glycols is retained. It has now been observed that the glycol recovered in accordance with the prior art and incorporated into de-icing agents has an only inadequate service life. It comprises a considerable fraction of glycol esters and other impurities which rules out or considerably hinders further use as aircraft de-icing agent.

The present invention therefore provides a method for reprocessing aircraft de-icing agents comprising glycol, in which
(1) the used aircraft de-icing agent, possibly contaminated with runway de-icing agent, is collected in a suitable device,
(2) the used aircraft de-icing agent is subsequently brought to a glycol content between 55 and 75% by weight without, or after only coarse, prior separation off of solid or suspended impurities, by expelling water at elevated temperature,
(3) the concentrated used aircraft de-icing agent obtained in this way is transported to a central reprocessing system, where it is subjected to a fine distillation, and where
(4) the glycol is produced as distillate of the fine distillation.

In a preferred embodiment, the glycols are glycols having 2 or 3 carbon atoms or diglycols having 4 to 6 carbon atoms, for example ethylene glycol, diethylene glycol or propylene glycol. Particular preference is given to propylene glycol and monoethylene glycol.

The used aircraft de-icing agent is passed from the place where the aircrafts are de-iced in the airport grounds via pipelines to a suitable container such as a storage tank or a collecting vessel. From there, it is brought to a suitable device in which the water can be partially expelled by heating. Here, observing CSB limits in the condensate, depending on local regulations, must be taken into consideration. The content of glycol in the residue from the water expulsion is between 55 and 75% by weight. The expulsion of water preferably takes place at a temperature of from 70 to 100° C.

Further processing of the concentrated, used aircraft de-icing agent then preferably no longer takes place at the site where this used aircraft de-icing agent was produced. Rather, this concentrate is taken from the site where it was produced to operating premises where a fine distillation can be carried out. The concentrate is subjected to a fine distillation in order to separate off the glycols from it.

Before being transported to the fine distillation or before the fine distillation, the concentrated used aircraft de-icing agent can be filtered. The concentrate obtained after the filtration comprises essentially glycol and water.

Prior to the fine distillation, the pH of the concentrate can be adjusted to at least 7, preferably at least 8 and in particular at least 9. For this, the concentrate is admixed with an alkaline agent until the stated pH is reached. Suitable alkaline agents are, for example, oxides and hydroxides of alkali metals and alkaline earth metals, such as, for example, calcium oxide, calcium hydroxide, sodium hydroxide or potassium hydroxide. The alkalis can be used in solid form or in solution, preferably aqueous solution.

The methods and devices for carrying out the fine distillation can correspond to those known in the prior art. The reprocessed glycol is obtained as distillate. The distillation bottom is discarded.

The method according to the invention is particularly suitable for used aircraft de-icing agents which comprise, besides water,
(a) 1 to 60% by weight, preferably 5 to 50% by weight, of glycols having 2 or 3 carbon atoms or diglycols having 4 to 6 carbon atoms,
(b) up to 0.8% by weight, preferably 0.05 to 0.5% by weight, of water-soluble polymers from the group of polyacrylates and polymethacrylates,
(c) 0.01 to 1% by weight of surfactants, preferably from the group of anionic surfactants, for example sulfonates such as olefinsulfonates and alkylbenzenesulfonates,
(d) 0.001 to 0.1% by weight of corrosion inhibitor, preferably from the group of triazoles.

The stated composition refers to the aircraft de-icing agent prior to its use.

The desired quality of the glycols obtained with the method according to the invention is stipulated by the following criteria:
a) a surfactant concentration of less than 100 ppm
b) a water content in accordance with DIN 51 777 of max. 1.0% by weight, preferably max. 0.6% by weight
c) a glycolic acid content of less than 100 ppm, preferably less than 20 ppm
d) a formic acid content of less than 100 ppm, preferably less than 20 ppm
e) a lactic acid content of less than 100 ppm, preferably less than 20 ppm
f) an acetic acid content of less than 100 ppm, preferably less than 20 ppm
g) a propionic acid content of less than 100 ppm, preferably less than 20 ppm

The desired content of glycol esters of the acids specified under c) to g) is at most as high as the content of the acids.

### EXAMPLES

Examples of a fine distillation of used aircraft de-icing agents are given below.

| | Example | | | | |
|---|---|---|---|---|---|
| | 1 | 2 | 3 | 4 | 5 |
| Water content (DIN 51777) % | 0.5 | 0.4 | 0.46 | n.d. | 0.6 |
| Surfactant content ppm | 66 | 66 | 136 | 82 | 54 |
| Glycolic acid ppm | <20 | <20 | <20 | <20 | <20 |
| Formic acid ppm | <20 | <20 | <20 | <20 | <20 |
| Lactic acid ppm | <20 | <20 | <20 | <20 | <20 |
| Acetic acid ppm | <20 | <20 | <20 | <20 | <20 |
| Propionic acid ppm | <20 | <20 | <20 | <20 | <20 |

1. A method for reprocessing aircraft de-icing agents comprising glycol, wherein the method comprises the steps of:
(1) collecting the used aircraft de-icing agent, possibly contaminated with runway de-icing agent, is in a suitable device,
(2) separating the collected used aircraft de-icing agent by expelling water at elevated temperature, to a glycol content between 55 and 75% by weight without, or after only coarse, prior separation off of solid or suspended impurities, to obtain a concentrated used aircraft de-icing agent,
(3) transporting the concentrated used aircraft de-icing agent to a central reprocessing system, where it is subjected to a fine distillation, and wherein
(4) glycol is produced as distillate of the fine distillation.

2. The method as claimed in claim 1, in which the separation off of solid or suspended impurities in step (2) involves a coarse filtration.

3. The method as claimed in claim 1, in which the aircraft de-icing agent comprises, prior to its use, besides water,
(a) 1 to 88% by weight, preferably 5 to 50% by weight, of glycols having 2 or 3 carbon atoms or diglycols having 4 to 6 carbon atoms,
(b) up to 0,8% by weight of water-soluble polymers from the group of polyacrylates and polymethacrylates,
(c) 0.01 to 1% by weight of surfactants, and
(d) 0.001 to 1% by weight of corrosion inhibitor.

4. The method as claimed in claim 1, where the expulsion of water takes place at a temperature of from 70 to 100° C.

5. The method as claimed in claim 1, where the glycol is propylene glycol or monoethylene glycol.
